(19) United States
(12) Patent Application Publication
(10) Pub. No.: US 2025/0222233 A1
(43) Pub. Date: Jul. 10, 2025

(54) MRI-COMPATIBLE TRANSSEPTAL NEEDLE

(71) Applicant: Imricor Medical Systems Inc., Burnsville, MN (US)

(72) Inventors: Joseph Thoen, Golden Valley, MN (US); Shawn J. Noble, Lakeville, MN (US); Scott Kimmel, St. Paul, MN (US)

(21) Appl. No.: 19/010,619

(22) Filed: Jan. 6, 2025

Related U.S. Application Data
(60) Provisional application No. 63/618,148, filed on Jan. 5, 2024.

(51) Int. Cl. A61M 25/06

(52) U.S. Cl. CPC A61M 25/0612

(57) ABSTRACT

A needle catheter that comprises: a handle; a shaft formed of a polymeric material and having a proximal end coupled to the handle; a needle tip formed of a non-ferrous metal material and comprising a tube with (i) a proximal end coupled to a distal end of the shaft, and (ii) a distal end with a multi-angle bevel defining a pointed structure with a side profile having a variable angle with respect to a center axis of the needle tip; and a central lumen extending through the handle, the shaft and the needle tip.

### CROSS-REFERENCE TO RELATED APPLICATIONS

The present application claims priority to and the benefit of U.S. Provisional Application Ser. No. 63/618,148 filed on Jan. 5, 2024. The content of this U.S. Provisional application is incorporated herein by reference in its entirety.

### FIELD OF THE INVENTION

The present solution is directed toward transseptal needle catheters. The needle catheters may be, for example, (1) used in conjunction with a dilator and sheath to gain access to the left atrium of the heart from the right atrium and/or (2) used to puncture the interatrial septum to facilitate crossing of the dilator, sheath, and additional devices across the septum.

### BACKGROUND

### Description of the Related Art

Methods for construction of transseptal needles and related devices intended for use within fluoroscopic environments are well known. A typical transseptal “kit” consists of a needle, dilator and sheath introducer. The sheath introducer has a lumen for delivery of a dilator, which then has its own lumen for delivery of a needle catheter to the septum. The needle catheter generally consists of a handle at the proximal end, a sharp beveled tip at the distal end, and a shaft connecting the two. The needle catheter is generally designed to be as rigid as possible since significant force must be delivered from the handle to the tip to puncture the septum. Often this means selecting a material such as stainless steel due to its rigidity. The major limitation of this design is that stainless-steel is not compatible for use within a magnetic resonance imaging (MRI) guided procedure due to radio frequency (RF) heating concerns. Performing invasive cardiovascular procedures such as transseptal crossing under MRI guidance offers significant advantages over fluoroscopy, as it eliminates exposure to radiation and enables visualization of soft tissue. Many materials that are considered MRI-compatible are polymeric, and therefore have much lower stiffness than the commonly used stainless steel.

Another limitation of current transseptal needles is the risk of skiving the dilator lumen while introducing the needle through the dilator. Without implementation of anti-skiving features, there is significant risk for the needle point to scrape the wall of the dilator lumen and expose the patient to particulate from the dilator lumen. Current transseptal needles use varying techniques to mitigate this risk. One configuration makes use of back bevels which intersect a main bevel to create a distinct point that is biased towards the center axis of the needle shaft, away from the dilator lumen wall. Another technique uses a stylet, delivered via a lumen within the needle, to guide the needle assembly down the center of the dilator lumen. Despite the use of these techniques, creation of particulate due to dilator skiving is still a concern associated with transseptal procedures.

### SUMMARY

The present disclosure concerns a needle catheter. The needle catheter comprises: a handle; a shaft formed of a polymeric material and having a proximal end coupled to the handle; a needle tip formed of a non-ferrous metal material and comprising a tube with (i) a proximal end coupled to a distal end of the shaft, and (ii) a distal end with a multi-angle bevel defining a pointed structure with a side profile having a variable angle with respect to a center axis of the needle tip; and a central lumen extending through the handle, the shaft and the needle tip.

The present disclosure also concerns a method for performing a medical procedure, comprising: advancing a needle tip of a needle catheter into a body of an individual, the needle tip formed of a non-ferrous metal material and comprising a tube with (i) a proximal end coupled to a distal end of a shaft formed of a polymeric material, (ii) a distal end with a multi-angle bevel defining a pointed structure with a side profile having a variable angle with respect to a center axis of the needle tip, and (iii) a central lumen extending therethrough; and puncturing an internal body part of the individual using the needle tip.

### BRIEF DESCRIPTION OF THE DRAWINGS

This disclosure is facilitated by reference to the following drawing figures, in which like numerals represent like items throughout the figures.

FIGS. 1A-1C (collectively referred to herein as “FIG. 1”) provide illustrations of a needle catheter.

FIG. 2 provides an illustration of a needle tip traversing a dilator lumen.

FIG. 3 provides an illustration of a needle assembly with a tubing that compresses when force is applied at a handle.

FIG. 4 provides an illustration of a transseptal needle handle.

FIG. 5 provides a transseptal needle with a nitinol tube for strain relief. The shaft tubing is fully constrained during crossing.

FIG. 6 provides an illustration that is useful for understanding an interaction between a dilator tip and needle collar.

FIG. 7 provides an illustration of a transseptal needle with a laser cut nitinol hypotube.

FIG. 8 provides a side view of a needle tip with a sharp multi-angled bevel.

FIG. 9 provides a front view of the needle tip shown in FIG. 8.

FIGS. 10-11 provide illustrations shown angles of a needle tip.

FIG. 12 provides an illustration showing a needle tip with a multi-angled bevel with a leading edge formed toward a center axis.

FIG. 13 provides an illustration showing a needle handle tab and a needle tip orientation relationship.

FIGS. 14A-14B (collectively referred to as “FIG. 14”) provide a flow diagram of an illustrative method for using the present solution in a medical context.

### DETAILED DESCRIPTION

It will be readily understood that the solution described herein and illustrated in the appended figures could involve a wide variety of different configurations. Thus, the following more detailed description, as represented in the figures, is not intended to limit the scope of the present disclosure, but is merely representative of certain implementations in various different scenarios. While the various aspects are presented in the drawings, the drawings are not necessarily drawn to scale unless specifically indicated.

Reference throughout this specification to features, advantages, or similar language does not imply that all of the features and advantages that may be realized should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout the specification may, but do not necessarily, refer to the same embodiment.

There remains a need to provide a transseptal needle catheter that is compatible with MRI guided procedures, while maintaining the rigidity required to facilitate transseptal crossing. There also remains a need to further reduce the risk of particulate generation by implementation of additional anti-skiving measures.

The present solution concerns an MRI-compatible transseptal needle catheter. The needle catheter has sufficient rigidity to facilitate crossing of the interatrial septum. Configurations may include various anti-skiving features to reduce the risk of particulate creation during typical use. The needle catheter consists of a handle, needle tip, and shaft constructed with materials that do not cause significant MRI induced RF heating. The needle catheter also features various anti-skiving measures.

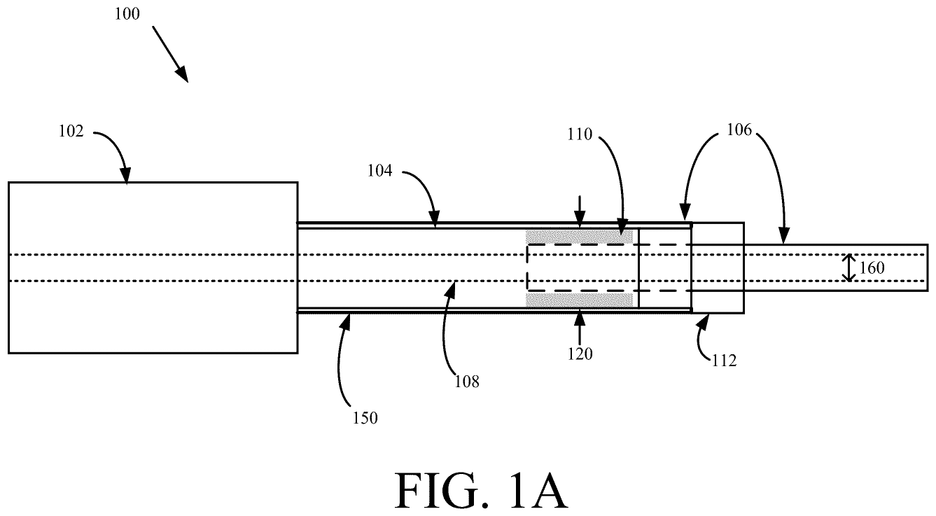

FIG. 1A provides an illustration of a needle catheter 100. The needle catheter 100 comprises a handle 102 and shaft 104 joined to the needle tip 106 via an adhesive 110. The adhesive can include, but is not limited to, an ultraviolet (UV) adhesive. Shaft 104 may be transparent or clear so that the UV adhesive can be cured therethrough. The handle, shaft and needle tip share a central lumen 108 to facilitate pressure monitoring. The handle and shaft can be made of any material selected in accordance with a given application. Such materials include, but are not limited to, polymeric material(s). Conventional needle catheters use metal for the shaft and not polymeric materials. Importantly, the use of polymeric material(s) for the handle and/or shaft renders these components free of significant MRI induced RF heating, as polymers are insulators and do not conduct RF currents. The handle can be made of the same or different material as that used for the shaft.

Needle tip 106 can be made of a non-ferrous metal to maximize hardness of the needle point and better facilitate crossing of the interatrial septum. Such non-ferrous materials include, but are not limited to, nitinol and/or other metals. Needle tips of conventional needle catheters do not comprise nitinol. Nitinol is selected because it is non-ferromagnetic, biocompatible, and produces fewer MRI imaging artifacts than other metallic materials such as stainless steel. The needle tip 106 may be formed of nitinol, for example, of an SE508 grade. The needle tip 106 may be formed entirely or partially of nitinol. In the partial nitinol scenario, the needle tip 106 comprises, for example, 50%-99% nitinol, 75%-99% nitinol, ≥75% nitinol, ≥80% nitinol, ≥85% nitinol, ≥90% nitinol, ≥95% nitinol, ≥96% nitinol, ≥97% nitinol, ≥98%. The remaining percentage can include another non-ferrous material. The present solution is not limited to the listed exemplary percentages of nitinol. The length 122 (shown in FIG. 1B) of needle tip 106 is selected to be relatively short to prevent accumulation of RF currents and therefore heating. If length 122 is too long, then the needle tip 106 may not be MR compatible. For example, to be MR compatible, length 122 may be between 1-2 cm. Lengths that may not be MR compatible include, for example, greater than 2 cm. The present solution is not limited to the listed lengths. Other lengths may be used in accordance with a given application.

More detailed illustrations of needle tip 106 are provided in FIGS. 1B-1C. Needle tip 106 consists of an inner tube 110 and an outer collar 112 that are joined together. Both the inner tube 110 and the outer collar 112 are formed of a non-ferrous material. The outer collar 112 can be coupled to the inner tube 110, or alternatively integrally formed with the inner tube 110 to provide a single part. The coupling of the outer collar 112 to the inner tube 110 can be achieved, for example, via a weld (e.g., laser weld), adhesive and/or friction fit.

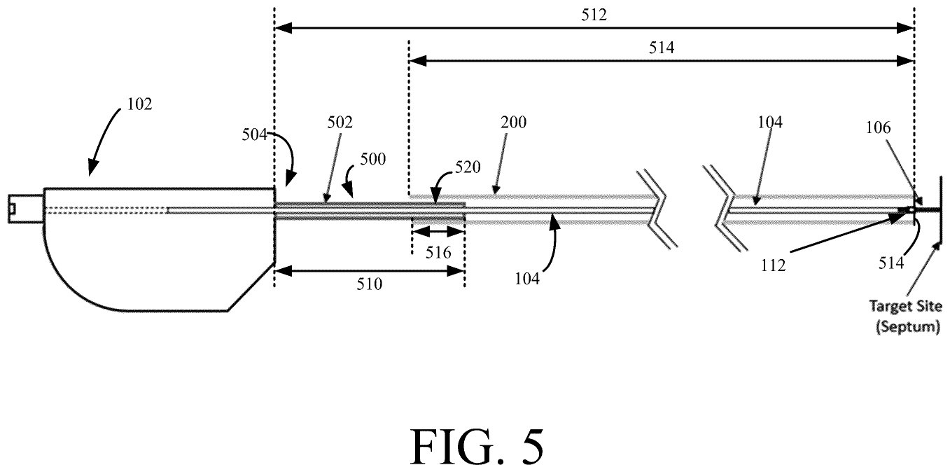

The inner tube 110 has a first portion 130 that extends out from the outer collar 112 in a first direction 140 and a second portion 132 that extends out and away from the outer collar 112 in an opposing second direction 142. The outer diameter 124 and length 118 of the first portion 130 are selected to facilitate coupling of the needle tip 106 to the shaft 104. The outer diameter 124 of the collar 112 is selected to be less than the inner diameter of the shaft 104 so that the first portion 130 is able to be slid or pushed into the shaft 104 while allowing sufficient clearance for the adhesive 110. The length 120 of the second portion 132 is selected to provide a given reveal length when the needle tip 106 is being used with a dilator. The reveal length 612 is shown in FIG. 6 as comprising the maximum length of the second portion 132 that is allowed to extend out and away from the dilator 600. The reveal length 612 can be defined by the following mathematical equation (1).

L₆₁₂=L₁₂₀−L₆₁₀ (1)

where L₆₁₂ represents the reveal length 612, L₁₂₀ represents length 120, and L₀₁₀ represents length 610. In some scenarios, the reveal length is selected to be 0.235″ to 0.315″. A distal end 114 of the inner tube 110 has an angled cut to create a sharp needle point.

The outer collar 112 has an outer diameter 140 that is the same as or similar to the outer diameter 120 (shown in FIG. 1) of the shaft 104. This allows the outer collar 112 to act as a stop for limiting the amount that the inner tube 110 can be inserted into the shaft 104. Outer collar 112 has other purposes which will become apparent as the discussion progresses. For example, outer collar 112 is configured to define the reveal length by limiting the total amount of the second portion 132 that can extend out and away from the dilator 600 during use.

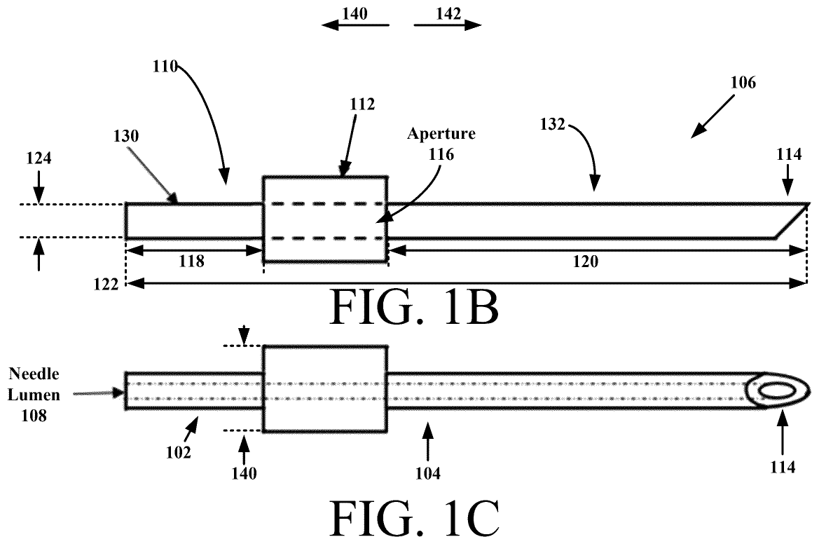

The outer collar diameter 140 is also selected to match or be less than an inner diameter 204 of a dilator lumen 200, as shown in FIG. 2. For example, the outer diameter 140 of the outer collar 112 is slightly less than the inner diameter 204 of the dilator lumen 200. Accordingly, the outer diameter 140 is, for example, ≤95%, ≤90%, ≤85%, and/or ≤80% of the inner diameter 204. The present solution is not limited to the particulars of this example. The size of the outer diameter 140 may be selected to ensure that the needle tip 106 is able to traverse the dilator lumen 200 and the needle tip's sharp pointed distal end 114 is less likely to contact the lumen wall 206 and potentially skive material. Decreasing the clearance between the collar 112 and the dilator lumen 200 as much as possible will better constrain alignment with the lumen axis, and further reduce the likelihood of skiving without increasing crossing force. Increasing the collar length has a similar effect, but because many dilators feature a fixed curve at the distal end 208, the collar length should remain short enough to minimize deflection while traversing the curve. The inner tube's outer diameter 124 is selected to be less than the outer collar diameter 140 to ensure there is clearance between the needle tip's sharp pointed distal end 114 and the dilator lumen 200. Accordingly, the outer diameter 124 is, for example, ≤65%, ≤60%, ≤55%, and/or ≤50% of the outer diameter 140. The present solution is not limited to the listed percentages of this example.

The catheter's shaft 104 is long and tubular in shape so that the needle tip 106 can be delivered through the entirety of the dilator lumen 200. The tubing of shaft 104 is sized and/or shaped to match the outer collar 112 as shown in FIG. 1A and FIG. 2. Shaft 104 can be made from any material selected in accordance with a given application. Such materials include, but are not limited to, polymeric materials, Zytel® nylon resin material (e.g., nylon 66), polyimide, and/or polyetherketone (PEEK).

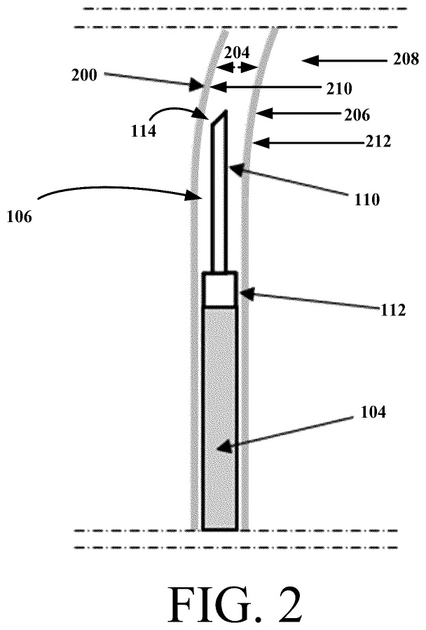

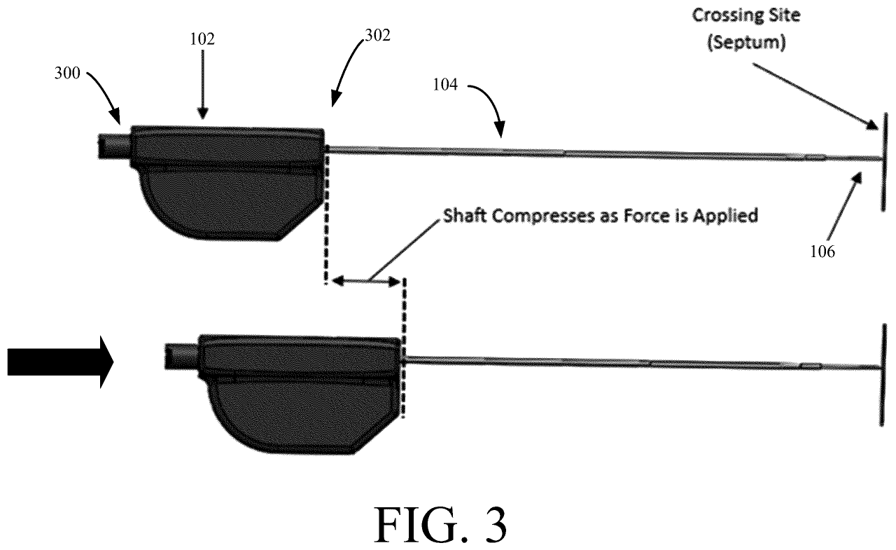

The material used for shaft 104 may be selected to be relatively stiff for minimizing compression of the tubing during crossing of the interatrial septum. Stated differently, shaft 104 is designed with a low compression rate to resist compression force when needle tip 106 is pushed. An illustration is provided in FIG. 3 showing this compression of the shaft when a pushing force is applied thereto. If the shaft tubing material is not sufficiently stiff, a large fraction of force applied at the handle 102 will be lost to tubing compression, and less force will reach the septum. Tubing compression therefore causes the handle 102 to be advanced further than expected as the compression must be overcome to provide enough force to cross the septum. Examples of tubing materials with sufficient stiffness to facilitate crossing include, but are not limited to, the Zytel® nylon resin material, polyimide, and PEEK.

The handle 102 can be formed of any material selected in accordance with a given application. Such materials include, but are not limited to, polymeric material(s). The handle 102 features a connector 400 at the proximal end 300, so the material should be rigid enough to maintain integrity of the connector. The connector 400 can include, but is not limited to, a luer connector. Any known or to be known luer connector can be selected and used here in accordance with a given application. The material should also have sufficient impact resistance and should be compatible with overmolding for ease of manufacture. Examples of materials that meet these requirements include, but are not limited to, a Grilamid® material and acrylonitrile butadiene styrene (ABS).

There is a potential for a stress riser to occur where the shaft tubing meets the handle 102, causing the shaft 104 to kink there during use. Thus, the needle catheter may optionally further include a strain relief 402 molded over the location where the shaft 104 meets the handle 102 to mitigate this concern.

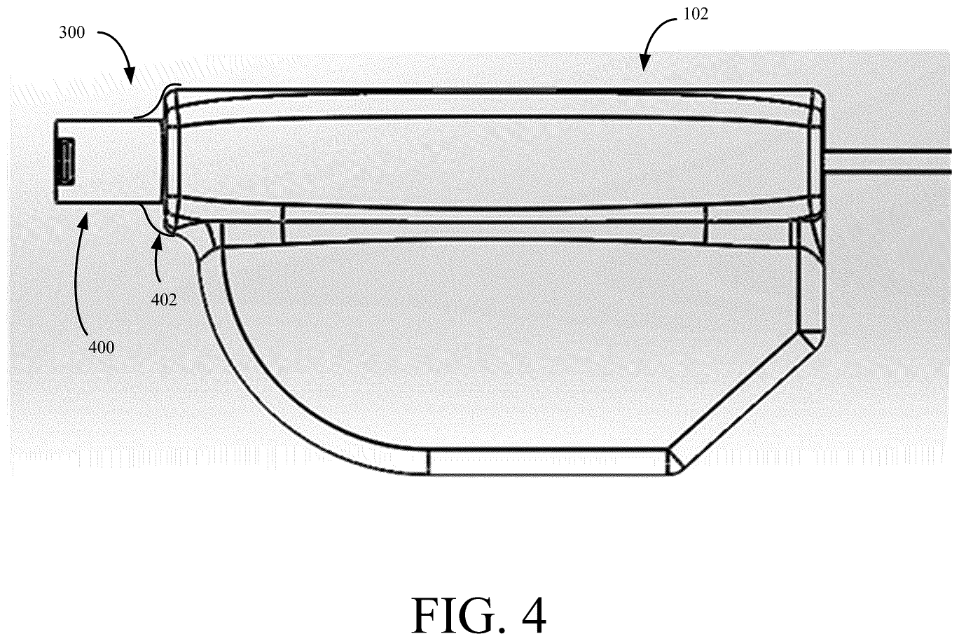

An illustration of a strain relief configuration is provided in FIG. 5. The strain relief 500 comprises a non-ferrous metal tube 502 covering a proximal portion of the shaft 104 at the shaft-handle transition 504. The non-ferrous metal tube 502 is coupled to the handle 102. This coupling can be achieved, for example, by over molding the non-ferrous metal tube 502 on the handle 102. The non-ferrous metal tube 502 can be made of, for example, nitinol and/or other non-ferrous metal. The non-ferrous metal tube 502 acts as a semi-rigid support for the shaft tubing 104, making it much less likely to buckle/kink during use. The non-ferrous metal tube 502 has a length 510 selected to ensure that all of the shaft tubing 104 is supported by either the dilator lumen 200 or the strain relief 500 during crossing, when the assembly sees peak force. The non-ferrous metal tube 502 is also designed such that a distal portion 520 thereof is inserted into the dilator lumen 200 by a certain amount 516. The length 512 is selected to limit the amount of compression of the inner tube 110 during use. For example, length 512 can be 0.5″-2.0″, 0.5″-1.5″, 0.5″-1.0″, 1.0″-2.0″, and/or 1.5″-2.0″. The present solution is not limited to the listed lengths. Length 512 may be selected to be long enough so that it extends into the dilator lumen 200 by a certain amount at least when the collar 112 is bottomed out inside the dilator tip.

As shown in FIG. 5, the length 510 of the strain relief 500 is greater than the difference between the usable length 512 of the needle shaft 104 and the overall length 514 of the dilator lumen 200. Thus, length 510 can be defined by the following mathematical equation (2).

L₅₁₀>>L₅₁₂−L₅₁₄ (2)

where L₅₁₀ represents length 510 of the strain relief 500, L₅₁₂ represents the usable length 512 of needle shaft 104, and L₅₁₄ represents the overall length 514 of the needle shaft 104 in the dilator lumen 200. So, the length of the needle shaft that is sticking out of the proximal end of the dilator is supported by the strain relief or hypotube.

When initially feeding the needle tip 106 into the dilator, some length of shaft tubing 104 distal to the strain relief 500 remains unsupported outside of the dilator lumen 200 and is susceptible to bending/buckling. The flexibility of the non-ferrous metal tube 502 allows it to relieve some of the bending strain present during feeding, making nitinol a suitable strain relief material over other metals like stainless steel.

Similar to the needle tip 106, the length of the strain relief 500 must be sufficiently short so as to prevent accumulation of RF currents and therefore heating. This length may be optimized so that it is long enough to provide sufficient strain relief, while short enough to be considered MRI-safe. In some scenarios, length 510 may be, for example, 2.5-3.0 inches. The present solution is not limited to the listed lengths. Other lengths may be used in accordance with a given application.

It should be noted that outer collar 112 is provided to limit the amount of the needle tip 106 that can extend out and away from the dilator tip 514. The manner in which this is achieved will now be explained in relation to FIG. 6. As shown in FIG. 6, dilator tip 514 has an inner channel with a variable width 602. The width 602 decreases in direction 604 towards the distal end 606. As such, the inner channel is partially defined by an angled sidewall 608. The outer collar 112 of the needle tip 106 is sized to engage the angled sidewall 608 when the needle tip 112 is pushed through the dilator 200. This engagement between the outer collar 112 and angled sidewall 608 centers the needle and limits the extent to which the needle tip 106 can extend out from the inner channel 620 of the dilator tip 514.

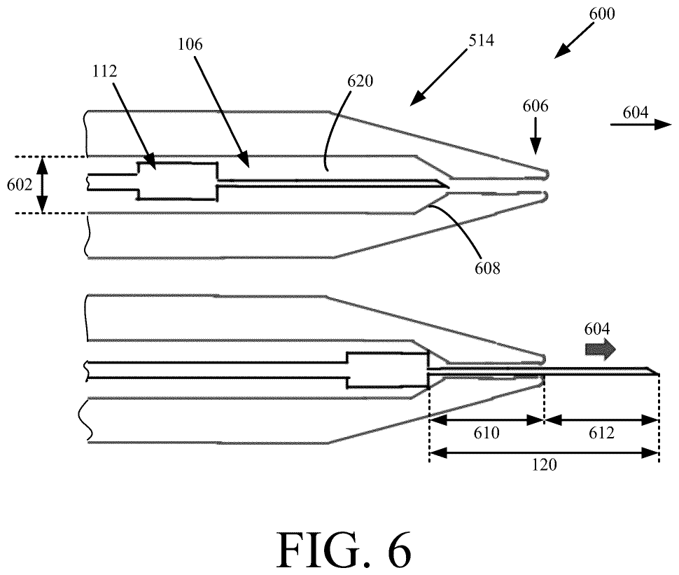

Another illustrative strain relief configuration is shown in FIG. 7. In FIG. 7, the strain relief 700 is formed of a non-ferrous metal tube 702 with a pattern 704 formed thereon. The pattern 704 can be formed, for example, via laser cutting. The pattern 704 can be a spherical pattern as shown in FIG. 7 or other pattern that increases the flexibility of the non-ferrous metal tube 702. The non-ferrous metal tube 702 may include, but is not limited to, nitinol. This configuration further increases the flexibility of the strain relief 700, allowing a higher fraction of bending stresses to take place within the non-ferrous metal tube 702, where the shaft 104 cannot kink easily since it is supported.

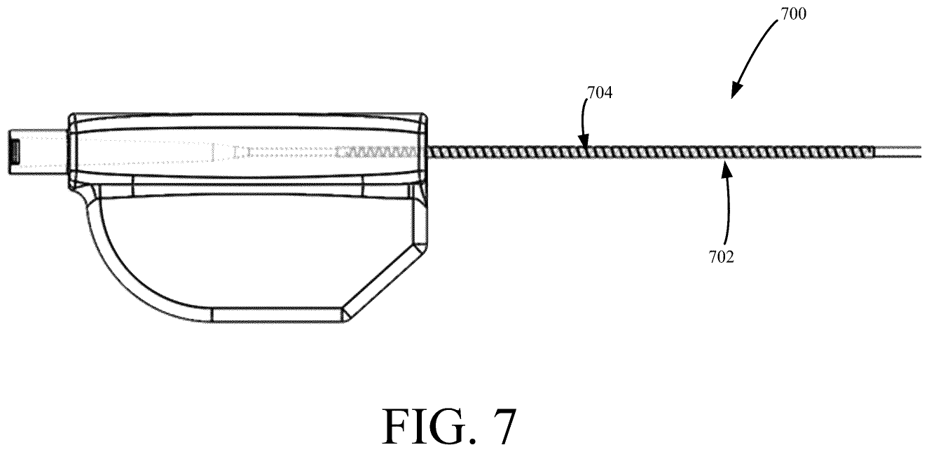

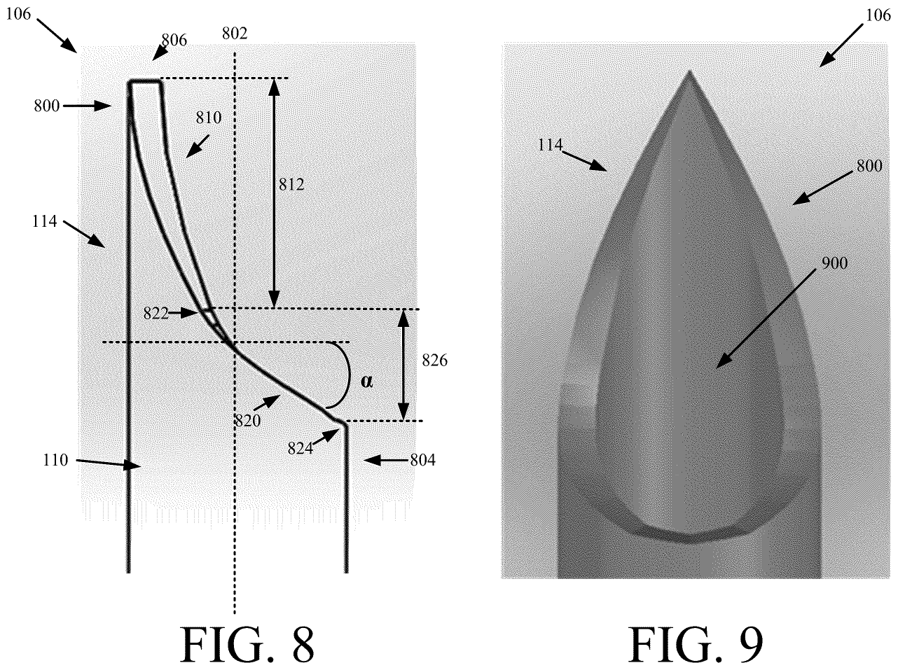

Referring now to FIGS. 8-9, illustrations of the distal end 114 of the needle tip 106 are provided. Conventional needle tips include a pointed structure which fully encompasses a central aperture. In contrast, the needle tip 106 of the present solution has an angled side profile such that the pointed structure 800 only partially encompasses the central aperture 900. The profile of the sharp needle point 800 is an important feature of the MRI compatible transseptal needle design. The shape of the needle point 800 may be optimized to have sufficient sharpness to puncture the septum, while not being so sharp that the dilator lumen 200 is easily skived.

The pointed structure 800 has a distal portion 810 and a proximal portion 820. The profile of the distal portion 810 has a spiral cut with a constant angle. The length 812 of the distal portion 810 is equal to or larger than the thickness of the object to be punctured by the needle tip 106 (e.g., a septum). For example, length 812 can be 0.020″-0.050″. The present solution is not limited in this regard. The constant angle can include, but is not limited to, N° with respect to the center axis 802 of the needle tip 106. N° can be any number selected in accordance with a given application. For example, in some scenarios, N° can be 18°, 19°, 20°, 21°, 22° or any number in the range 18°-22°. The present solution is not limited to the listed numbers for N°.

The profile of the proximal portion 820 has a continuously decreasing angle from a first point 822 to a second point 824 or increasing angle from point 824 to point 822. The decrease or increase in angle can be linear or non-linear. For example, in some scenarios, the angle can decrease or increase 5° every fifty thousandths of an inch from 20° to a primary bevel angle α. The primary bevel angle α can include, but is not limited to, 40° with respect to the center axis 802 of the needle tip 106. The present solution is not limited to the listed angles. The length 826 of the proximal portion 820 may be selected to minimize the chances of bucking of the needle tip responsive to applied force thereto when puncturing the object (e.g., a septum). For example, length 826 can be 0.010″-0.025″. The present solution is not limited in this regard.

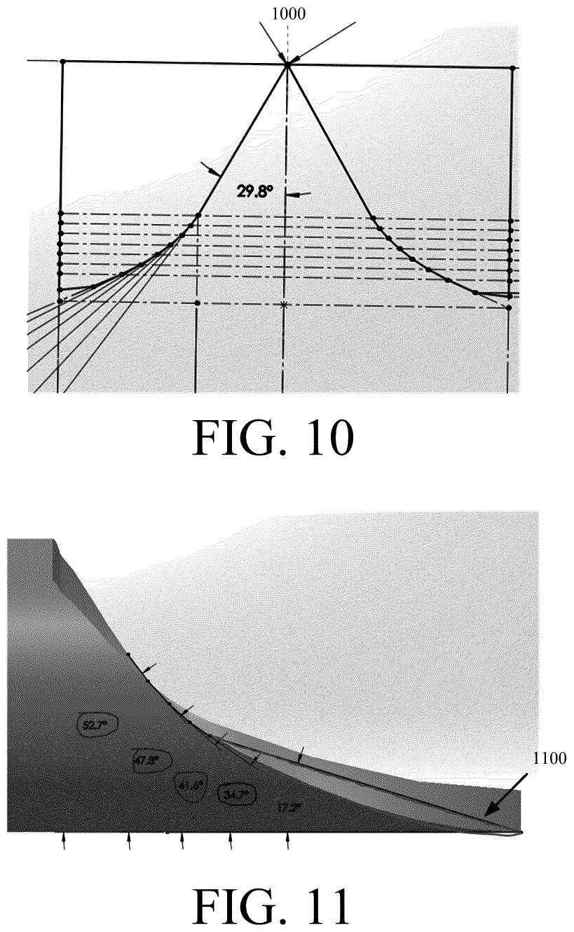

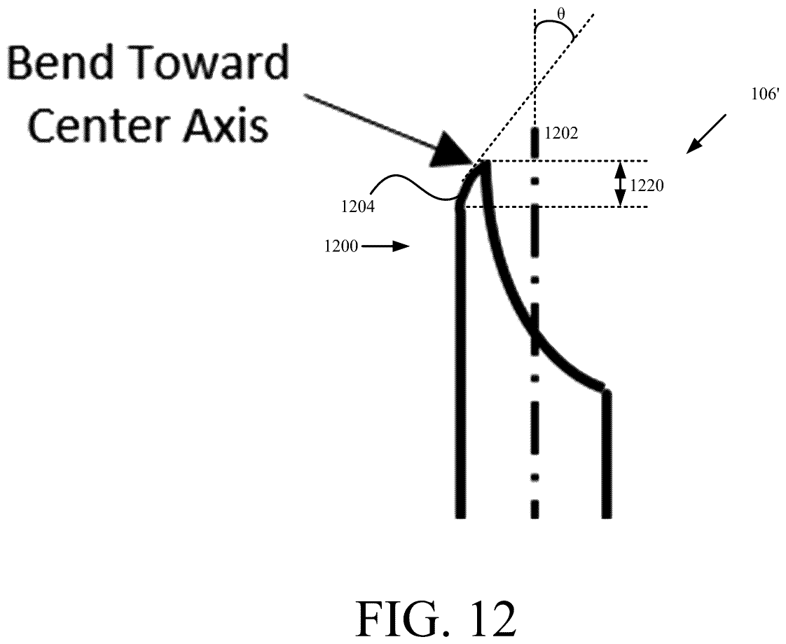

FIGS. 10-11 provide illustrations of another spiral cut for a needle tip. As shown in FIG. 10, the constant angle is 29.8° with respect to the center axis 1000 of the needle tip 106. The decrease or increase in angle of the proximal portion is non-liner as shown in FIG. 11. For example, the first angle decrease is by 4.9° (i.e., 52.7°-47.8°) while a next angle decrease is by 6.2° (i.e., 47.8°-41.6°), and so on. The present solution is not limited to the particular nonlinear configuration shown FIGS. 10-11.

It should be noted that, when the particular nonlinear configuration of FIGS. 10-11 is mapped onto the circular needle tip, the apparent angle becomes 17.2° at the distal portion with respect to the center axis of the needle tip. The angle of the cut then increases by some amount in increments of ˜4.5°-17.5° every 0.002″ depending on where the tool is at in the cut profile. The angle is always increasing by some amount every 0.002″.

The fact that this profile is laser cut with a spiral path adds complexity to defining the angle (as seen by outlined region 1100 in FIG. 11, the angle is not an exact match) but it can generally be said that the angle is increased after the distal portion in order to reduce the overall length of the point. When a flat pattern is mapped onto a certain outer diameter, this then defines the helix angle or pitch of the spiral cut. The purpose is to take advantage of reduced puncture force due to sharper angle, while minimizing risk of the point bending.

The above-described needle profile strikes a reasonable balance between tip sharpness and skiving risk. However, because the MRI compatible transseptal needle features a polymeric shaft with relatively low stiffness (e.g., as compared to that of stainless steel), the needle sharpness may be increased to reduce overall crossing force, since this will lead to less compression of the tubing. One configuration of the needle point profile that increases sharpness features a multi-angled bevel as shown in FIGS. 8-9 that has a shallow angle at the tip 800 of the needle, but gradually increases in angle until the bevel terminates at the opposite side 804 of the inner tube 110. The sharper angle at the distal point 806 of the bevel decreases the force needed to cross, and tapering the bevel angle reduces its overall length which increases the needle point's bending resistance. The tradeoff to this configuration is that increasing the sharpness of the leading edge inherently increases risk of skiving.

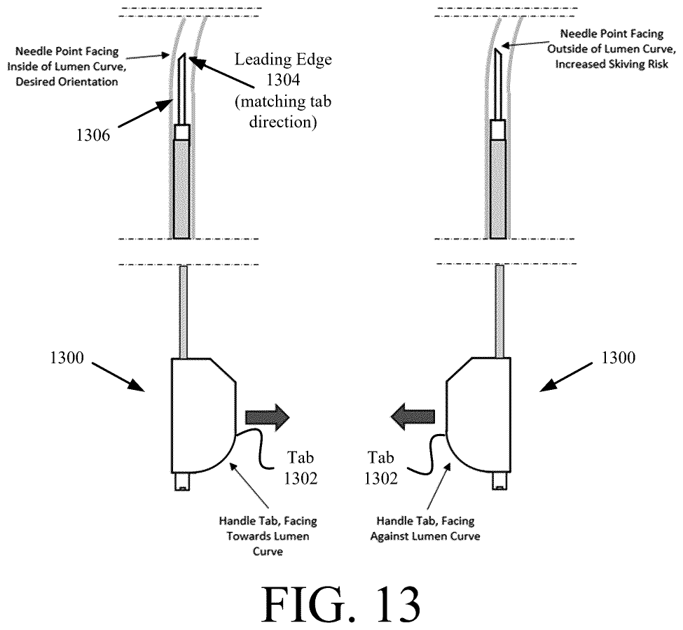

Referring to FIG. 12, there is provided an illustration of another configuration for the needle tip. As shown in FIG. 12, the needle tip 106′ has a distal end 1200 that has been cut with an identical profile to tip 800 of FIGS. 8-9. The distal end 1200 may be formed to bias the leading edge 1204 towards the center axis 1202 of the needle tip 106′. The super-elastic and shape memory properties of nitinol enable the unmodified bevel to be bent towards the center to a considerable degree without plastically deforming, and the shape memory properties ensure that this shape will be retained after heat treatment. Biasing the leading edge 1204 towards the center axis 1202 moves the needle point away from the dilator lumen wall 206, decreasing the risk of skiving. In some scenarios, the angle θ is 10°-20° and length 1220 is selected to be 0.010″-0.015″. The present solution is not limited in this regard.

When used with a dilator featuring a curved distal end 208, the orientation of the needle point/bevel has a significant impact on skiving risk. Orienting the needle tip 106′ so the leading edge 1204 is positioned closest to the inside wall 210 of the curved lumen path prevents the leading edge/needle point from contacting the outside wall 212 of the curved lumen path, where the angle of contact would be higher and have increased risk of skiving.

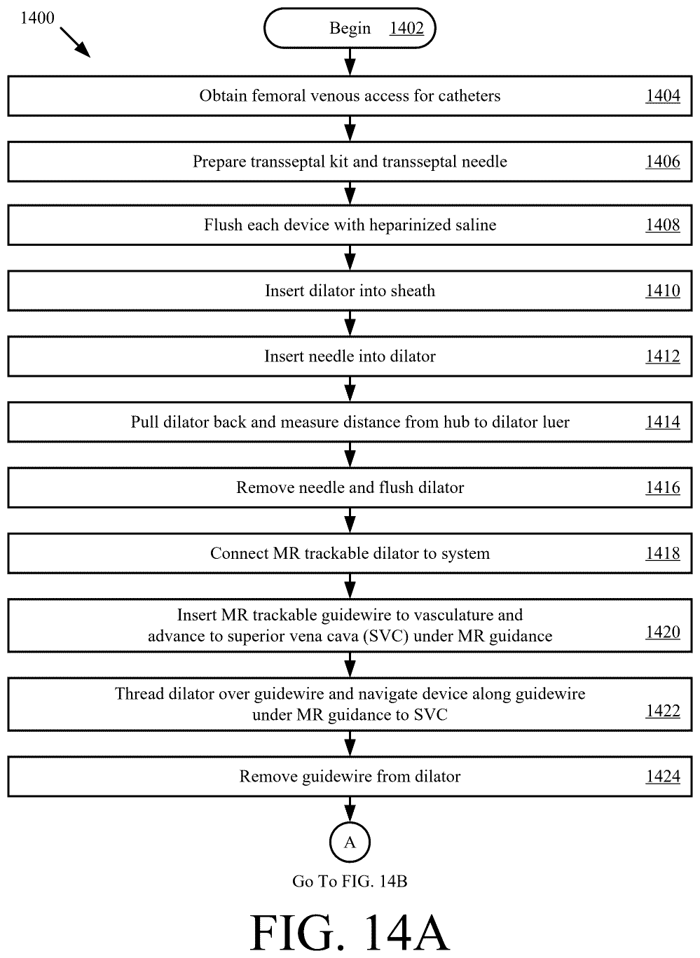

Referring now to FIG. 13, there is provided an illustration of a handle 1300 with a tab 1302. The tab 1302 is aligned with the leading edge 1304 of the needle tip 1306. This provides the user knowledge of the needle point alignment after insertion into the dilator, so(s) he may control its orientation during feeding to minimize skiving.

When used as the connecting member between the handle 1300 and needle tip 1306, certain polymer materials have insufficient torque response to maintain the orientation of the needle tip 1306 relative to the handle's tab 1302. Frictional forces within the dilator lumen can cause the needle tip 1306 to twist during feeding, and without sufficient torsional rigidity the needle tip 1306 will no longer be aligned with the handle's tab 1302. Thus, shaft materials may be used with higher torsional rigidity to better mitigate this concern. These materials can include, but are not limited to, PEEK, polyimide, or other rigid polymers. A torque response may be increased more significantly by introducing braid reinforcement 150 to the shaft tubing 104. Multiple configurations are possible including, but not limited to, a PEEK shaft reinforced with Kevlar braid, or a polyimide shaft reinforced with PEEK or LCP braid.

In view of the forgoing, the present solution comprises: (i) an MRI compatible transseptal needle (system level, everything is compatible); and (ii) a metal needle tip designed that remains MR compatible despite using metal. In order to make the needle catheter MRI compatible, polymer tubing may be used for shaft instead of metal. Using a polymer shaft tubing instead of metal reduces performance of the catheter (tubing compresses/twists more easily). In order to compensate for this performance loss, certain features were designed: (a) metal strain relief, with potential flexible cut pattern; (b) a sharper tip with multi angled bevel; and (c) a stiffened polymer material through material selection (PEEK, polyimide), or braid reinforcement. Skiving of dilator lumen is a concern present for all transseptal systems. A novel method to reduce skiving is forming the nitinol needle tip towards the center axis.

The present solution also concerns implementing systems and methods for performing a medical procedure, such as an MRI guided procedure (e.g., MRI guided biopsy and/or radiotherapy). For example, the needle catheters may be (1) used in conjunction with a dilator and sheath to gain access to the left atrium of the heart from the right atrium of the heart and/or (2) used to puncture the interatrial septum to facilitate crossing of the dilator, sheath, and additional devices across the septum.

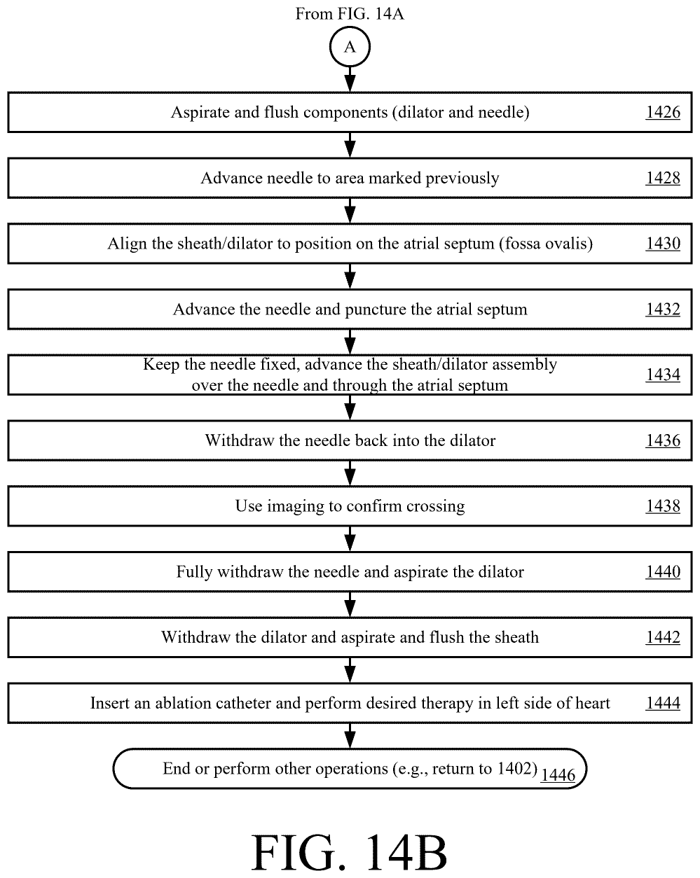

FIG. 14 provides a flow diagram of an illustrative method 1400 for facilitating a medical procedure using the above described transseptal needle catheter(s). Method 1400 begins with 1402 and continues to 1404 where femoral venous access is obtained for catheters (e.g., needle catheter 100 of FIG. 1). Any known or to be known techniques to obtain femoral venous access for catheters can be used here. Next in 1406, a transseptal kit and transseptal needle (e.g., needle 106 of FIG. 1) are prepared. Each device is flushed with heparinized saline in block 1408. It should be noted here that the transseptal needle inside diameter (e.g., diameter 160 of FIG. 1) is open in order to allow injecting or aspiration of fluid.

Subsequently in blocks 1410-1412, a dilator (e.g., dilator 600 of FIG. 6) is inserted in a sheath and the transseptal needle is inserted into the dilator lumen (e.g., dilator lumen 200 of FIG. 2). This step is relevant to FIG. 13. It is important the needle is inserted the right way otherwise it can damage the inside of the dilator lumen, or worse skive material away from the dilator lumen and introduce a thrombosis into the body. In FIG. 12, the needle tip 106′ is bent forward to further this and yet have an even sharper needle for the procedure. The dilator is pulled back in block 1414. A distance is also measured from a hub to a dilator luer. The dilator has a hard stop in the bottom that stops the needle from protruding too much therefrom. This measurement is for physician reference to know when the needle is fully extended out of the dilator lumen. The polymer nature of the body of the needle creates a spongy feeling that can be confusing if not noted. In block 1416, the needle is removed from the dilator. The dilator is then flushed.

Thereafter, operations of blocks 1418-1424 are performed. These operations involve: connecting an MR trackable dilator (e.g., dilator 600 of FIG. 6) to the system; insert an MR trackable guidewire to vasculature of a patient; advance the guidewire to superior vena cava (SVC) of the patient under MR guidance; thread the dilator over the guidewire; navigate the dilator along the guidewire under MR guidance to SVC; and remove the guidewire from the dilator. These operations can be performed in any known or to be known manner.

Method 1400 then continues with the operations of block 1426 of FIG. 14B. As shown in FIG. 14B, block 1426 involves flushing the dilator and needle (e.g., needle 106 of FIG. 1). The needle is advanced to the previously marked area inside the patient (i.e., the SVC). The sheath or dilator is aligned to a position in the atrial septum (fossa ovalis) of the patient, as shown by block 1430. The needle is advanced in block 1432 and used to puncture the atrial septum of the patient. The needle profile shown in FIGS. 8-10 lowers the puncture force of the atrial septum. The needle profile shown in FIG. 12 maintains a lower puncture force and pushes in the sharp part to mitigate skiving while inserting the needle through the dilator.

Once the atrial septum is punctured, method 1400 continues to block 1436 where the needle is kept in a fixed position. The sheath and dilator assembly is advanced over the needle and through the atrial septum. Next in block 1436, the needle is withdrawn to a predetermined position.

Imaging is used in block 1438 to confirm crossing. Any known or to be known technique for confirming crossing based on imaging can be used here. The handle luer 400 may also allow it to be connected to a pressure sensor to monitor intracardiac pressure through the channel 160 inside the needle catheter but not limited to other compatible diagnostic devices. The needle is withdrawn further in block 1440, and the dilator is aspirated. The dilator is withdrawn in block 1442, and the sheath is aspirated and flushed. An ablation catheter is inserted in block 1444 so that a desired therapy can be performed in the left side of the heart. Once the therapy is completed, method 1400 continues to block 1446 where it ends or other operations are performed. The other operations can include, but are not limited to, returning to block 1402 of FIG. 14A.

The described features, advantages and characteristics disclosed herein may be combined in any suitable manner. One skilled in the relevant art will recognize, in light of the description herein, that the disclosed systems and/or methods can be practiced without one or more of the specific features. In other instances, additional features and advantages may be recognized in certain scenarios that may not be present in all instances.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

Although the systems and methods have been illustrated and described with respect to one or more implementations, equivalent alterations and modifications will occur to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application. Thus, the breadth and scope of the disclosure herein should not be limited by any of the above descriptions. Rather, the scope of the invention should be defined in accordance with the following claims and their equivalents.

Without excluding further possible embodiments, certain example embodiments are summarized in the following Clauses.

Clause 1: A needle catheter, comprising: a handle; a shaft formed of a polymeric material and having a proximal end coupled to the handle; a needle tip formed of a non-ferrous metal material and comprising a tube with (i) a proximal end coupled to a distal end of the shaft, and (ii) a distal end with a multi-angle bevel defining a pointed structure with a side profile having a variable angle with respect to a center axis of the needle tip; and a central lumen extending through the handle, the shaft and the needle tip.

Clause 2: The needle catheter according to Clause 1, wherein the needle tip further comprises an outer collar disposed on the tube.

Clause 3: The needle catheter according to any preceding Clauses, wherein a first portion of the tube extends in a first direction from a first side of the outer collar and a second portion of the tube extends in an opposing second direction from a second side of the outer collar, the second portion comprising the pointed structure of the needle tip.

Clause 4: The needle catheter according to any preceding Clauses, wherein the first portion of the tube is inserted in the distal end of the shaft such that the first side of the outer collar abuts the shaft.

Clause 5: The needle catheter according to any preceding Clauses, wherein the outer collar has a dual purpose of (i) limiting an amount that the first portion of the tube can be inserted into the shaft and (ii) limiting an amount that the second portion can extend out and away from a dilator tip.

Clause 6: The needle catheter according to any preceding Clauses, wherein the pointed structure only partially encompasses a center aperture of the needle tip.

Clause 7: The needle catheter according to any preceding Clauses, wherein the pointed structure comprises a distal portion with a constant angle with respect to a center axis of the needle tip, and a proximal portion with an angle that decreases with respect to a center axis of the needle tip that starts at a point located where the distal portion meets the proximal portion and ends at a point located on an outer surface of the needle tip.

Clause 8: The needle catheter according to any preceding Clauses, wherein a length of the distal portion is equal to or greater than a thickness of an object to be punctured by the needle tip.

Clause 9: The needle catheter according to any preceding Clauses, wherein the constant angle is between 10° and 30° with respect to the center axis of the needle tip.

Clause 10: The needle catheter according to any preceding Clauses, wherein the angle of the proximal portion of the pointed structure decreases in a linear manner or a non-linear manner.

Clause 11: The needle catheter according to any preceding Clauses, wherein the angle of the proximal portion of the pointed structure has a primary bevel angle value of 40°.

Clause 12: The needle catheter according to any preceding Clauses, wherein a length of the proximal portion of the pointed structure is selected to minimize bucking of the needle tip responsive to an applied force when puncturing an object.

Clause 13: The needle catheter according to any preceding Clauses, wherein the needle tip is MRI compatible.

Clause 14: The needle catheter according to any preceding Clauses, wherein the needle tip comprise a non-ferrous metal.

Clause 15: The needle catheter according to any preceding Clauses, wherein the non-ferrous metal comprises nitinol.

Clause 16: The needle catheter according to any preceding Clauses, wherein the needle catheter comprises an MRI compatible transseptal needle.

Clause 17: The needle catheter according to any preceding Clauses, wherein the distal end of the needle tip is bent towards the center axis of the needle tip.

Clause 18: The needle catheter according to any preceding Clauses, further comprising a strain relief that is disposed around the proximal end of the shaft and configured to reduce bucking of the shaft during use of the needle catheter.

Clause 19: The needle catheter according to any preceding Clauses, wherein the strain relief comprises a tube formed of a non-ferrous metal.

Clause 20: The needle catheter according to any preceding Clauses, wherein the non-ferrous metal comprises nitinol.

Clause 21: The needle catheter according to any preceding Clauses, wherein the strain relief is over molded on the handle.

Clause 22: The needle catheter according to any preceding Clauses, wherein a portion of the strain relief is disposed in a dilator lumen.

Clause 23: The needle catheter according to any preceding Clauses, wherein a length of the strain relief is greater than a difference between a length of a dilator lumen and a length of the shaft.

Clause 24: The needle catheter according to any preceding Clauses, wherein a pattern is formed on an outer surface of the strain relief so as to increase a flexibility of the strain relief.

Clause 25: The needle catheter according to any preceding Clauses, wherein the pattern comprises a spherical pattern.

Clause 26: The needle catheter according to any preceding Clauses, wherein the shaft comprises a braid reinforcement.

Clause 27: The needle catheter according to any preceding Clauses, wherein the needle tip comprises a multi-angled tip.

Claim 28: Use of the needle catheter according to any preceding Clauses.

Clause 29: A method for performing a medical procedure, comprising: using the needle catheter according to any preceding Clauses to perform the medical procedure.

Clause 30: A method for performing a medical procedure, comprising: advancing a needle tip of a needle catheter into a body of an individual, the needle tip formed of a non-ferrous metal material and comprising a tube with (i) a proximal end coupled to a distal end of a shaft formed of a polymeric material, (ii) a distal end with a multi-angle bevel defining a pointed structure with a side profile having a variable angle with respect to a center axis of the needle tip, and (iii) a central lumen extending therethrough; and puncturing an internal body part of the individual using the needle tip.

Clause 31: The method of Clause 29, further comprising advancing the needle tip through a dilator lumen without damaging an inside of the dilator lumen or skiving material away from the dilator lumen.

Clause 32: The method of any preceding method Clauses, wherein the needle catheter comprises the needle catheter of any preceding Clause 1-27.

The breadth and scope of this disclosure should not be limited by any of the above-described example embodiments, but should be defined only in accordance with the following claims and their equivalents.

We claim:

1. A needle catheter, comprising:
a handle;
a shaft formed of a polymeric material and having a proximal end coupled to the handle;
a needle tip formed of a non-ferrous metal material and comprising a tube with (i) a proximal end coupled to a distal end of the shaft, and (ii) a distal end with a multi-angle bevel defining a pointed structure with a side profile having a variable angle with respect to a center axis of the needle tip; and
a central lumen extending through the handle, the shaft and the needle tip.

2. The needle catheter according to claim 1, wherein the needle tip further comprises an outer collar disposed on the tube.

3. The needle catheter according to claim 2, wherein a first portion of the tube extends in a first direction from a first side of the outer collar and a second portion of the tube extends in an opposing second direction from a second side of the outer collar, the second portion comprising the pointed structure of the needle tip.

4. The needle catheter according to claim 3, wherein the first portion of the tube is inserted in the distal end of the shaft such that the first side of the outer collar abuts the shaft.

5. The needle catheter according to claim 3, wherein the outer collar has a dual purpose of (i) limiting an amount that the first portion of the tube can be inserted into the shaft and (ii) limiting an amount that the second portion can extend out and away from a dilator tip.

6. The needle catheter according to claim 1, wherein the pointed structure only partially encompasses a center aperture of the needle tip.

7. The needle catheter according to claim 1, wherein the pointed structure comprises a distal portion with a constant angle with respect to a center axis of the needle tip, and a proximal portion with an angle that decreases with respect to a center axis of the needle tip that starts at a point located where the distal portion meets the proximal portion and ends at a point located on an outer surface of the needle tip.

8. The needle catheter according to claim 7, wherein a length of the distal portion is equal to or greater than a thickness of an object to be punctured by the needle tip.

9. The needle catheter according to claim 7, wherein the constant angle is between 10° and 30° with respect to the center axis of the needle tip.

10. The needle catheter according to claim 7, wherein the angle of the proximal portion of the pointed structure decreases in a linear manner or a non-linear manner.

11. The needle catheter according to claim 7, wherein the angle of the proximal portion of the pointed structure has a primary bevel angle value of 40°.

12. The needle catheter according to claim 7, wherein a length of the proximal portion of the pointed structure is selected to minimize bucking of the needle tip responsive to an applied force when puncturing an object.

13. The needle catheter according to claim 1, wherein the needle tip is MRI compatible.

14. The needle catheter according to claim 13, wherein the needle tip comprise a non-ferrous metal.

15. The needle catheter according to claim 13, wherein the non-ferrous metal comprises nitinol.

16. The needle catheter according to claim 1, wherein the needle catheter comprises an MRI compatible transseptal needle.

17. The needle catheter according to claim 1, wherein the distal end of the needle tip is bent towards the center axis of the needle tip.

18. The needle catheter according to claim 1, further comprising a strain relief that is disposed around the proximal end of the shaft and configured to reduce bucking of the shaft during use of the needle catheter.

19. The needle catheter according to claim 18, wherein the strain relief comprises a tube formed of a non-ferrous metal.

20. The needle catheter according to claim 19, wherein the non-ferrous metal comprises nitinol.

21. The needle catheter according to claim 18, wherein the strain relief is over molded on the handle.

22. The needle catheter according to claim 18, wherein a portion of the strain relief is disposed in a dilator lumen.

23. The needle catheter according to claim 18, wherein a length of the strain relief is greater than a difference between a length of a dilator lumen and a length of the shaft.

24. The needle catheter according to claim 18, wherein a pattern is formed on an outer surface of the strain relief so as to increase a flexibility of the strain relief.

25. The needle catheter according to claim 24, wherein the pattern comprises a spherical pattern.

26. The needle catheter according to claim 1, wherein the shaft comprises a braid reinforcement.

27. The needle catheter according to claim 1, wherein the needle tip comprises a multi-angled tip.

28. A method for performing a medical procedure, comprising:
advancing a needle tip of a needle catheter into a body of an individual, the needle tip formed of a non-ferrous metal material and comprising a tube with (i) a proximal end coupled to a distal end of a shaft formed of a polymeric material, (ii) a distal end with a multi-angle bevel defining a pointed structure with a side profile having a variable angle with respect to a center axis of the needle tip, and (iii) a central lumen extending therethrough; and
puncturing an internal body part of the individual using the needle tip.

29. The method according to claim 28, further comprising advancing the needle tip through a dilator lumen without damaging an inside of the dilator lumen or skiving material away from the dilator lumen.
